(19) United States
(12) Patent Application Publication
(10) Pub. No.: US 2026/0322786 A1
(43) Pub. Date: Oct. 15, 2026

(54) DISTRIBUTED STORAGE AND MANAGEMENT OF DATA

(71) Applicant: 3SHAPE A/S, Copenhagen K (DK)

(72) Inventors: Kasper EGDØ, Copenhagen K (DK); André Glasius TISCHER, Copenhagen K (DK); Morten Riiskjær BOYSEN, Copenhagen K (DK)

(73) Assignee: 3SHAPE A/S, Copenhagen K (DK)

(21) Appl. No.: 18/998,847

(22) PCT Filed: Jul. 31, 2023
(86) PCT No.: PCT/EP2023/071184

§ 371 Date: Jan. 27, 2025

(30) Foreign Application Priority Data
Jul. 29, 2022 (EP) 22187702.0

(51) Int. Cl. H04L 9/40; G06F 21/60

(52) U.S. Cl. CPC H04L 63/0428; G06F 21/602; H04L 63/166

(57) ABSTRACT

A method and system for distributed storage and management of data may include storing a data abstract that includes one or more structured data entities, each including at least three node pairs. The first node pair including a hash of a first node in a second node pair, a unique identifier, a digital signature, and a data key. The first node of the second node pair including a hash a first node in a third node pair and a hash of the second node of the second node pair. The second node of the second node pair is encrypted with entity relation node key and includes a summary of data included in an encrypted data node and a data decryption key for the encrypted data node. The third node pair including the encrypted data node having data encrypted using the data encryption key, and an encrypted data file.

### FIELD

The present disclosure relates to methods, systems, and computer program products for distributed storage and management of data. More particularly, the present disclosure relates to storing sensitive data in a system where knowledge of and access to sensitive data is controlled via directed acyclic graphs.

### BACKGROUND

Computer networks are holding highly sensitive data across numerous different computer systems requiring numerous levels of access and security. Identifying, sending, and accessing this data in an efficient and secure manner is an increasingly complex task. This is especially true for medical data. Medical data has various legal and ethical protections to protect the privacy of this data. Such protections may include how and where the data is stored, when and by whom the data may be viewed, where and why the data may be sent, and when and how the data may be deleted. Previously this medical data was stored and retrieved primarily for reading by a human (such as a medical professional). However, other computer systems and personnel may need at least partial access to the data based upon the introduction of new technologies, such as automated and semi-automated diagnostics, computer modeling, computer-aided design of various medical devices and prosthetics, etc. Dental data is an example field of this medical data which would benefit from the ability to store and share sensitive data in distinct levels and packages so that these new technological functions can be performed quickly, securely, and in compliance with various regulations.

Peer-to-peer (P2P) storage systems combine the storage capacity of a network of storage devices (peers) into a common pool of storage space to store and share content among the peers. P2P systems provide data persistence and availability agnostic of the capabilities of each individual peer in a decentralized environment. Current P2P methods and technologies, including distributed data storage, require each device in the system to have a significant storage capacity as each device stores a full copy of the relevant database and/or requires the same data to be transmitted multiple times between and among the devices of the P2P storage system. There are current P2P systems that avoid having each device within the P2P system having a copy of the entire database, but in such systems local caching of data is generally required. Also, in such current P2P systems, each device within the P2P system must know that the piece of data exists and where it is stored within the system. For example, if a device needs a piece of data, the device would have to request the piece of data and store the piece of data locally on the device for use. Current data storage systems also fail to distinguish between knowing of the data stored in the storage system versus actually having the data stored in the storage system. For example, current methods and technologies propagate data to every device in the storage system by providing a full copy of a database to each device, e.g. a distributed network. Further, current methods and technologies require data stored in a storage system to be transmitted between and amongst all the devices of the storage system multiple times resulting in inefficient transfer and storage of data. Lastly, current data storage systems including P2P storage systems are not offline tolerant, especially when it comes to data collisions, e.g., when the same piece of data is updated offline simultaneously by different devices. In such instances of data collision, current data storage systems may save the same data as different versions, save the data under different filenames, or requires a user to select which version of the data should be stored and discards the other. Thus, there is a need for methods and systems for more efficient storage, management, and access to stored data in a P2P storage system.

### SUMMARY

Described herein are various systems and methods that solve the above-mentioned needs for efficient, secure, and compliant handling and transferring of sensitive data, such as medical and/or dental data. Systems and methods described herein allow for greater accessibility and sharing of information among properly credentialed computer systems. The manner and location of storage and retrieval are based, at least in part, on the credentials of the computer systems and users and well as the types of data.

A method for distributed storage and management of data is disclosed. The method includes storing, by a computing device in a network, a data abstract interfaced with a database, the data abstract storing one or more structured data entities, each of the one or more structured data entities including at least three node pairs including: a first transport header node paired with a commit node, the first transport header node including at least a blob reference of the commit node, one of the blob references of the commit node including a hash of the commit node and size of the one of the blob references of the commit node and a second blob reference of the first transport header including a hash of a second transport header node and size of the second blob reference of the first transport header, the commit node including a unique identifier, a digital signature, and a data key, the data key being a relation node key encrypted with an entity key specific to an associated structured data entity of the one or more structured data entities; the second transport header node paired with an encrypted relation node encrypted with the relation node key, the second transport header including at least a blob reference of the encrypted relation node, one of the blob references of the encrypted relation node including a hash of a third transport header node and size of the one of the blob references of the encrypted relation node and a second blob reference of the second transport header including a hash of the encrypted relation node and size of the second blob reference of the second transport header, the encrypted relation node including a summary of data included in an encrypted data node and a data decryption key for the encrypted data node; the third transport header node paired with the encrypted data node and an encrypted data file, the third transport header node including at least a blob reference of the data node, the blob reference of the data node including at least a size of the encrypted data node and a hash of the encrypted data node, wherein the encrypted data node includes encrypted data related to the associated structured data entity encrypted using the data encryption key; transmitting, by the computing device, a request for the first transport header node and the paired commit node of a specific structured data entity of the one or more structured data entities; and receiving, by the computing device, the first transport header node and the paired commit node of the specific structured data entity

A system for distributed storage and management of data is disclosed. The system including a memory of a processor in a network configured to: store a data abstract interfaced with a database, the data abstract storing the one or more structured data entities, each of one or more structured data entities including at least three node pairs including: a first transport header node paired with a commit node, the first transport header node including at least a blob reference of the commit node, the blob reference of the commit node including a hash of the commit node and a hash of a second transport header node, the commit node including a unique identifier, a digital signature, and a data key, the data key being a relation node key encrypted with an entity key specific to an associated structured data entity of the one or more structured data entities; the second transport header node paired with an encrypted relation node encrypted with the relation node key, the second transport header including at least a blob reference of the encrypted relation node, the blob reference of the encrypted relation node including a hash of a third transport header node and a hash of the encrypted relation node, the encrypted relation node including a summary of data included in an encrypted data node and a data decryption key for the encrypted data node; the third transport header node paired with the encrypted data node and an encrypted data file, the third transport header node including a blob reference of the data node, the blob reference of the data node including at least a size of the encrypted data node and a hash of the encrypted data node, wherein the encrypted data node includes encrypted data related to the associated structured data entity encrypted using the data encryption key; a transmitting device of the processor configured to transmit a request for the first transport header node and the paired commit node of a specific structured data entity of the one or more structured data entities; and a receiving device of the processor configured to receive the first transport header node and the paired commit node of the specific structured data entity.

According to an embodiment, a computer program product for distributed storage and management of data is disclosed. The computer program product embodied in a non-transitory computer readable medium, the computer program product comprising computer readable program code being executable by a hardware data processor to cause the hardware data processor to perform method for distributed storage and management of data, as disclosed in one or more embodiments of this disclosure.

### BRIEF DESCRIPTION OF THE DRAWING FIGURES

The scope of the present disclosure is best understood from the following detailed description of exemplary embodiments when read in conjunction with the accompanying drawings. Included in the drawings are the following figures:

FIG. 1 illustrates a high-level system architecture for distributed storage and management of data in accordance with exemplary embodiments;

FIG. 2 illustrates an example producer device 102a in accordance with exemplary embodiments;

FIG. 3A illustrates an example first transport header node and commit node of a structured data entity in accordance with exemplary embodiments;

FIG. 3B illustrates an example second transport header node and relation node of a structured data entity in accordance with exemplary embodiments;

FIG. 3C illustrates an example third transport header node and data node of a structured data entity in accordance with exemplary embodiments;

FIG. 4 illustrates and example structured data entity in accordance with exemplary embodiments;

FIGS. 5A-5C is a flowchart illustrating a method for distributed storage and management of data in accordance with exemplary embodiments; and

FIG. 6 is a block diagram illustrating a computer system architecture in accordance with exemplary embodiments.

### DETAILED DESCRIPTION

As discussed above, current methods and technologies of data storage including distributed data storage require significant storage capacity with each node in the storage system having a full copy of the database requiring the same data to be transmitted multiple times between and among the devices of the storage system, and/or integrate knowledge of the data domain being modelled. Exemplary embodiments of the methods and systems provided herein provide a peer-to-peer storage engine with support for local indexing, e.g., projection indexing, and search data stored as structured data entities including, but not limited to a dental scan, a designed crown, a set of settings, etc. The structured data entities in the methods and systems provided herein overcome limitations on the actual structure of data being stored, e.g., data tables, data arrays, linked lists, stacks, queues, hash tables, trees, heaps. graphs, etc. The methods and systems provided herein allow data to split into multiple data chunks, which may be used in multiple structured data entities while at the same time enabling the advantages of P2P storage systems and directed acyclic graphs. The methods and systems provided herein consist of peers with different access controls by defining the functions and capabilities of the peers based on a peer type. Therefore, a more efficient and secure network for handling, storing, and distributing sensitive data, e.g., dental patient data, is provided by the methods and systems disclosed herein. The methods and systems provided herein provide an offline tolerant system in which data collisions, e.g., when the same piece of data is updated offline simultaneously by different devices, are permitted and stored within the directed acyclic graph. Also, the data of the methods and systems provided herein exists in two manners: first, certain peers may have knowledge about the existing data of structured data entities, but cannot access the actual data itself, which is encrypted, and second, certain peers may have knowledge about the existing data and have access to the data itself via the user of one or more cryptographic keys.

### System Overview for Distributed Storage and Management of Data

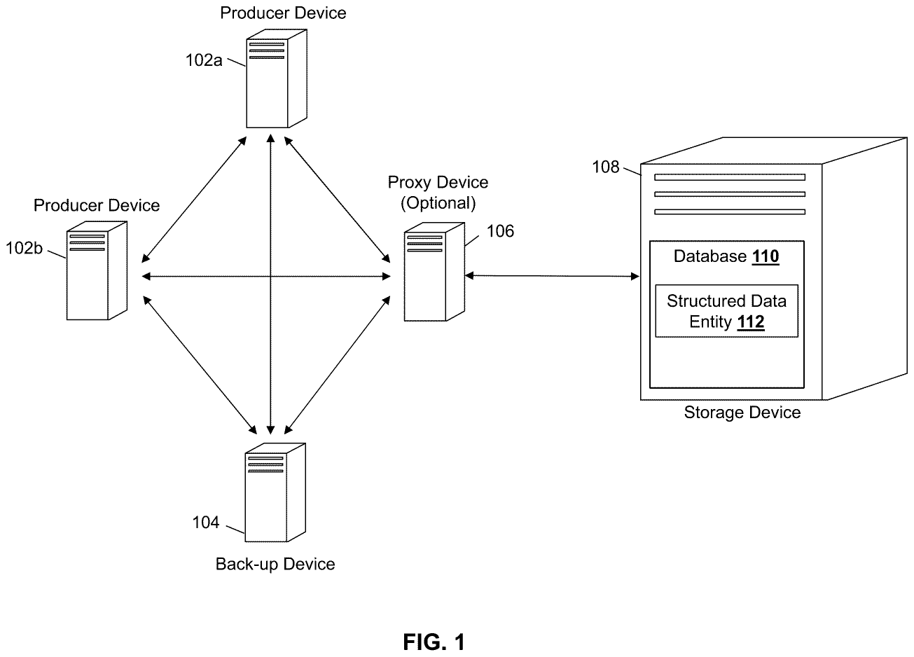

FIG. 1 illustrates system 100 for distributed data storage and management of data in accordance with exemplary embodiments. In some embodiments, the data storage and management of data is performed to protect sensitive data, such as medical data or more specifically dental data.

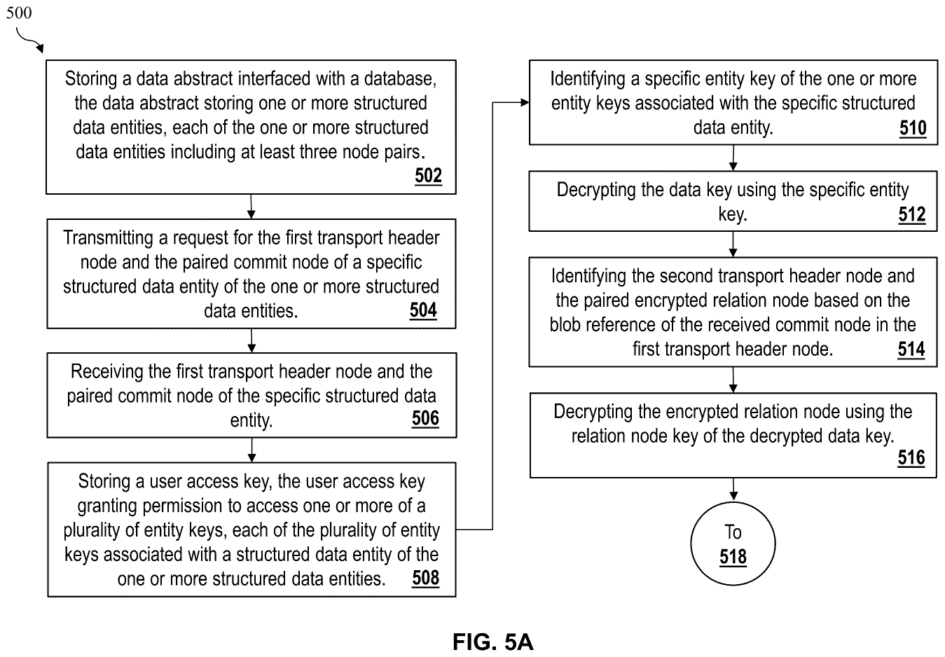

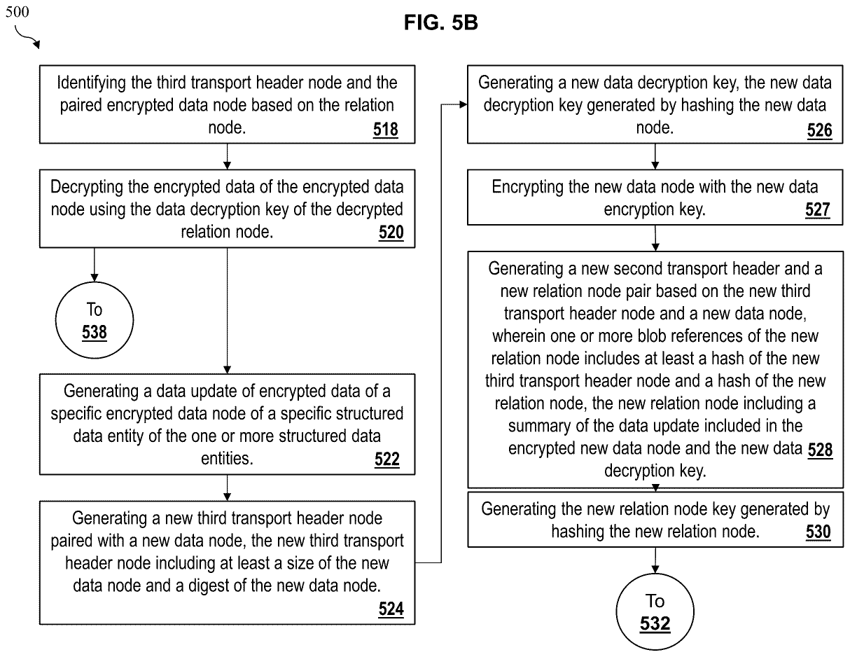

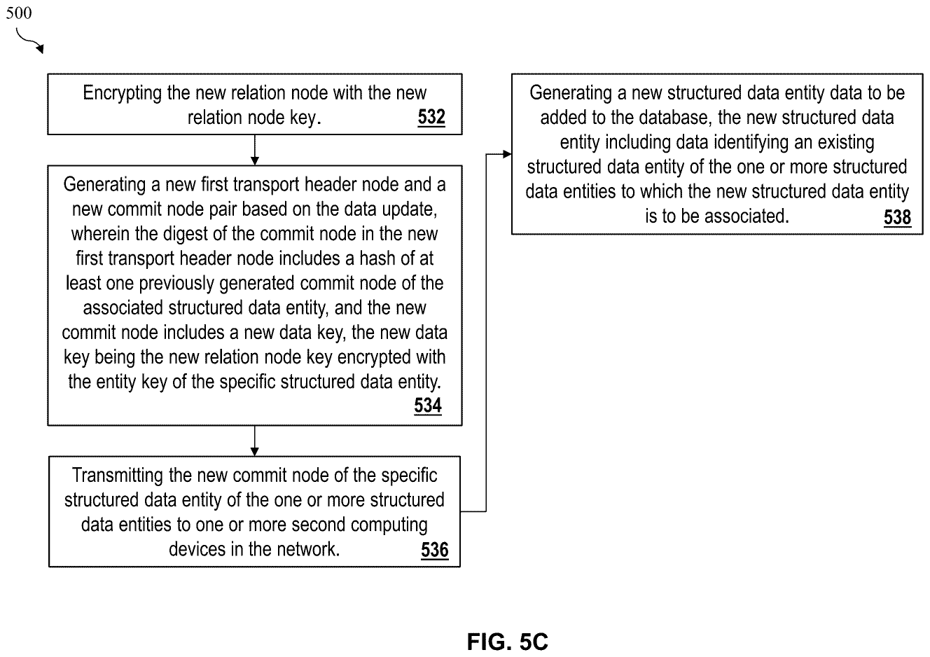

The system 100 includes one or more peers operating one or more devices including one or more producer devices 102a-102b, a back-up device 104, a proxy device 106, and a storage device 108 for generating, accessing, managing, and storing data of one or more structured data entities. The one or more peers can be divided into two general peer types: peers with a user-facing application, e.g., the one or more producer devices 102a-102b and the back-up device 104, that can read and write data in the system 100 and background peers that store and transfer data but do not have data read or write permissions, e.g., the back-up device 104, the proxy device 106, and the storage device 108. User-facing peers may run in the context of a specific user, e.g., a registered user. Background peers may be set up without a specific user and will not understand nor be able to read most of the data passing through them, for example, due to encryption. Both user-facing peers and background peers have access to a full data history, e.g., data size, of the one or more structured data entities, but not all peers have access to the actual underlying data as will be described in more detail below. Peer types will be discussed in more detail below with reference to FIGS. 1 and 5. While producer devices 102a-102b, a single back-up device 104, and a single proxy device 106 are illustrated as being a part of the system 100 in FIG. 1, it can be appreciated that any number of producer devices 102, back-up devices 104, and proxy devices 106 can be a part of the system 100 including none, one, or more than one.

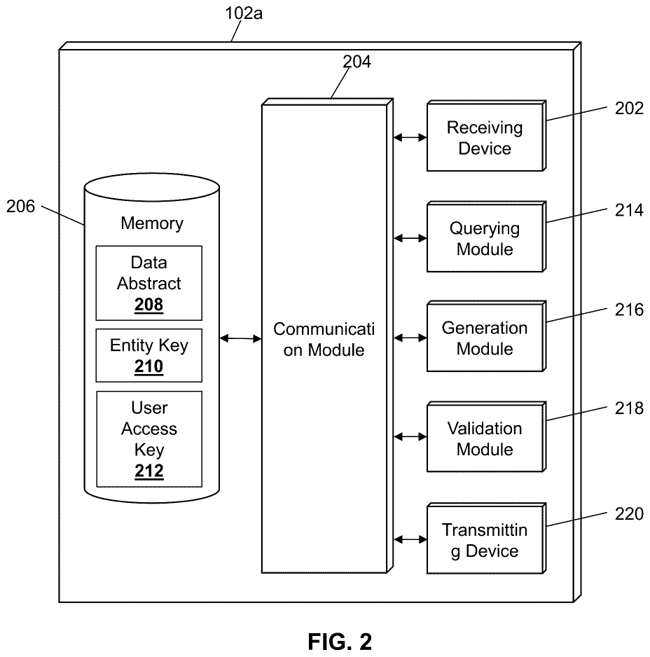

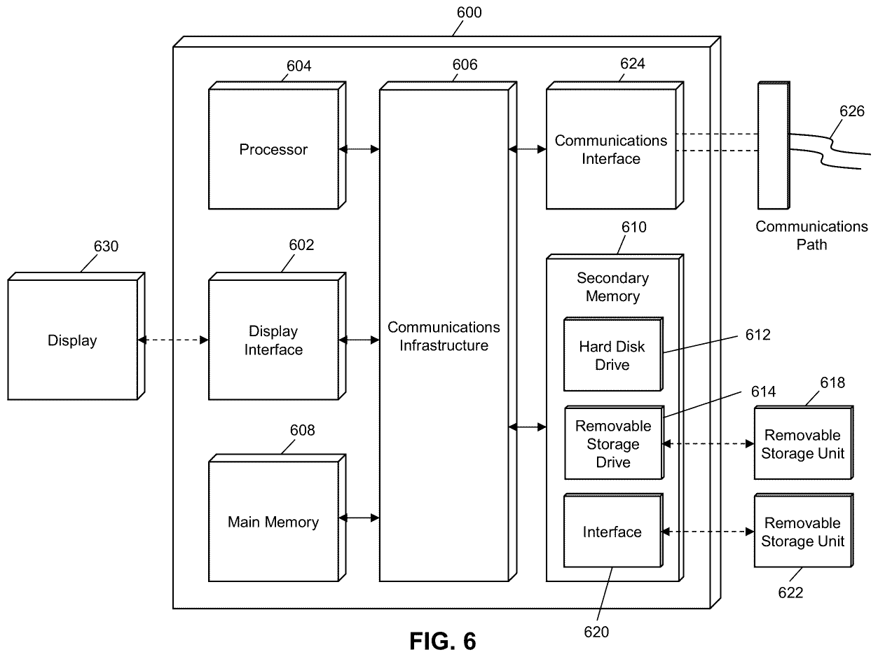

The one or more producer devices 102a-102b may be a server, a desktop computer, a notebook, a laptop computer, a tablet computer, a handheld device, a smart-phone, a thin client, or any other electronic device or computing system capable of generating, storing, compiling, organizing, and/or displaying audio, visual, and/or textual data and receiving and sending that data to and from other computing devices, such as the back-up device 104, the proxy device 106, and/or the storage device 108. For example, the one or more producer devices 102a-102b may be any type of electronic device or computing system specially configured to perform the functions discussed herein, such as the computing system 600 illustrated in FIG. 6. In an embodiment, the producer devices 102a-102b generate data associated one or more structured data entities, e.g., a dental patient scan, and transmit that data to the storage device 108. For example, the one or more producer devices 102a-102b are devices located in a dental office such as, but not limited to, a dental imaging device, a dental design device, etc. For example, the producer device 102a may be an intra-oral scanning device, such as, but not limited to, a TRIOS series Intraoral Scanner by 3Shape AS or Lab dental scanner, such as, but not limited to, E series lab scanners by 3Shape A/S, or any other intra-oral scanner, e.g. as disclosed in WO 2002/16867 “Method and Apparatus for Three-Dimensional Optical Scanning of Interior Surfaces” filed 24 Aug. 2001, WO 2010/048960 A1 “Scanner with Feedback Control” filed on 28 Oct. 2009, WO 2012/083967 A1 “Optical System in 3D Focus Scanner” filed on 21 Dec. 2011, WO 2018/172257 “3D Scanner System with Handheld Scanner” filed 19 Mar. 2018, U.S. Pat. No. 11,076,146 “Focus Scanning Apparatus” filed on 19 Mar. 2021, which are herein incorporated by reference in their entirety. An example producer device 102a is discussed in more detail with reference to FIG. 2. Further, while two producer devices 102a-102b are illustrated, it can be appreciated that any number of producer devices 102 may be a part of the system 100 including less than or more than two. Also, the producer devices 102a-102b may be located in one or more physical locations. For example, the producer device 102a may be a dental imaging device, e.g., an intra-oral scanner, located in a first room of a dental office, and the producer device 102b may be a dental design device, e.g., a computing device user to design a dental crown, located in a second room of a dental office or in a separate remote office, and the producer device 102c (not illustrated) may be a computing device located at a manufacturer location, e.g., a dental crown manufacturer, etc.

The back-up device 104 may be a server, a desktop computer, a notebook, a laptop computer, a tablet computer, a handheld device, a smart-phone, a thin client, a USB drive, or any other electronic device or computing system capable of storing, compiling, organizing, and or displaying audio, visual, and/or textual data and receiving and sending that data to and from other computing devices, such as the one or more producer devices 102a-102b, the proxy device 106, and/or the storage device 108. For example, the back-up device 104 may be any type of electronic device or computing system specially configured to perform the functions discussed herein, such as the computing system 600 illustrated in FIG. 6. In an embodiment, the back-up peer 104 may store data associated with a structured data entity that has been deleted from the system 100 for a specified period of time after the structured data entity that has been deleted so that the deleted structured data entity may be restored in the system 100. While only a single back-up device 104 is illustrated it can be appreciated that any number of back-up devices 104 may be a part of the system 100. For example, if the producer devices 102a-102b are located in multiple physical locations there may be a back-up device 104 located in each physical location or a single location may include more than one back-up device 104. Further, the back-up device 104 may be a temporary device in the system 100. For example, the back-up device 104 may be a USB drive that is only part of the system 100 when it is inserted into another device of the system 100, e.g., the one or more producer devices 102a-102b, the proxy device 106, the storage device 108, etc.

When a backup server is added to the network, the peers on the network will automatically discover the new backup server. This will allow the peers, through some proper UI, to configure the backup server using e.g. 3DD. Then the back-up server is configured, the peers will start to offload all their data to the backup server. In other words, installing a backup server is as simple as plugging in its network cable, power cord and then configuring it remotely.

The proxy device 106 is an optional device 106 in the system 100. The proxy device 106 may be a server, a desktop computer, a notebook, a laptop computer, a tablet computer, a handheld device, a smart-phone, a thin client, or any other electronic device or computing system capable of storing, compiling, organizing, and or displaying audio, visual, and/or textual data and receiving and sending that data to and from other computing devices, such as the one or more producer devices 102a-102b, the back-up device 104, and/or the storage device 108. For example, the proxy device 106 may be any type of electronic device or computing system specially configured to perform the functions discussed herein, such as the computing system 600 illustrated in FIG. 6. In an exemplary embodiment, the proxy device 106 is a temporary storage device that acts as an intermediary between the one or more producer device 102a-102b, the back-up device 104, and the storage device 108. For example, the one or more producer devices 102a-102b may be located in a dental office and the proxy device 104 may act as an intermediary between the producer devices 102a-102b and a communications network, e.g., the Internet. In the system 100, the proxy device 106 offloads data from the producer devices 102a-102b and transfers that data to the storage device 108 such that the producer devices 102a-102b can be taken offline, moved or shut down. The proxy device 106 may also be a cache of data retrieved by the producer devices 102a-102b in the system 100 enabling the producer devices 102a-102b to retrieve data through the proxy device 106 from the storage device 108 once without having to communicate with the storage device 108 in subsequent retrieval of the same data. The proxy device 106 reduces the amount of connections in the system 100 such that each producer device 102 does not need a connection to the storage device 108 or to each of the other producer devices 102.

The storage device 108 includes a database 110. The storage device 108 can be deployed on one or more nodes, e.g., storage or memory nodes, within a cloud storage system, or one or more processing-capable nodes such as a server computer, desktop computer, notebook computer, laptop computer, tablet computer, handheld device, smart-phone, thin client, or any other electronic device or computing system capable of storing, compiling, and/or processing data and computer instructions, and receiving and sending that data to and from other devices, such as the producer devices 102a-102n, the back-up device 104, and/or the proxy device 106. The storage device 108 may be located in the same or a different physical location as one or more of the producer devices 102a-102b, the back-up device 104, and/or the proxy device 106. For example, if the storage device 108 is a cloud storage device, the storage device 108 will be located in different physical location from the remaining devices of the system 100, e.g. in a different State or Country, and communication with the remaining devices of the system 100 via the Internet or any other suitable communication network. Further, while only a single storage device 108 is illustrated, it can be appreciated that any number of storage device 108 can be a part of the system 100 and that each of the storage devices 108 may be located in a different physical location. For example, the system 100 may include two storage devices 108, a first storage device 108 located in the same physical location as the producer devices 102a-102b, e.g., in a dental office, and a second storage device 108 located remote from the physical location of the producer devices 102a-102b. In an embodiment having more than one storage device 108, each of the storage devices 108 may the entire database 110 or each of the storage devices 108 may store different portions of the database 110.

The database 110 can be any suitable storage configuration, such as, but not limited to, a relational database, a structured query language (SQL) database, a distributed database, an object database, a file storage database, a blob storage database, etc. Further, the storage device 108 may be a cloud storage device and/or system. Suitable configurations and storage types will be apparent to persons having skill in the relevant art. In an exemplary embodiment, the devices of the system 100 do not store a full copy of the database 110, but rather each device in the system 100 has access to a copy of a data abstract, e.g., the data abstract 208, of the database 110, which is discussed in more detail below with reference to FIG. 2. The database 110 includes structured data entity 112. While only a single structured data entity 112 is illustrated, it can be appreciated that any number of structured data entities 112 can be part of the database 110. The database 110 includes the full history and content of each structured data entity 112. Each structured data entity 112 includes the history and content associated with a data file. For example, a structured data entity 112 may be, but is not limited to, a dental scan, e.g., from an intra-oral imaging device, a designed crown, a set of settings, etc. Each structured data entity 112 includes at least three pairs of nodes that form a directed acyclic graph (DAG).

As the content of the data stored and replicated is opaque to the system itself, the applications using the system needs to search for specific entities. The system may provide a mechanism of “projections” which search of those projections. The projection is done by user-supplied code (e.g., controlling software), which is called or otherwise activated by the system. The data used for searching is stored un-encrypted (or partially un-encrypted) to allow the database to search. However, the database itself must be encrypted at rest, so data is not leaked or otherwise accessed in an unauthorized manner. Some embodiments may reduce this by “attaching” a user specific database containing the projected data for this specific user.

When a user system can access and decrypt data, the user system supplies one or more custom projections that allows a user to extract the data they need to index in a form they find most suitable. These projections may be limited to the entities the user system understands and can deserialize.

It should be appreciated that embodiments of the invention separate knowledge of data from the data itself. Thus, it is possible to create projections and searchable data by partial traversal and transfer of the DAGs. The result is that blobs/data not needed for the projection are not transferred. This means an entity can be projected and made searchable without transferring all the blobs the entity consists of.

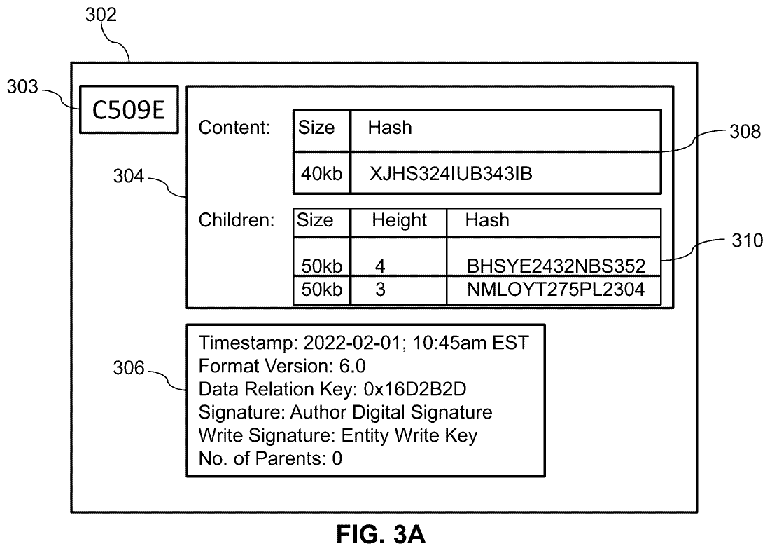

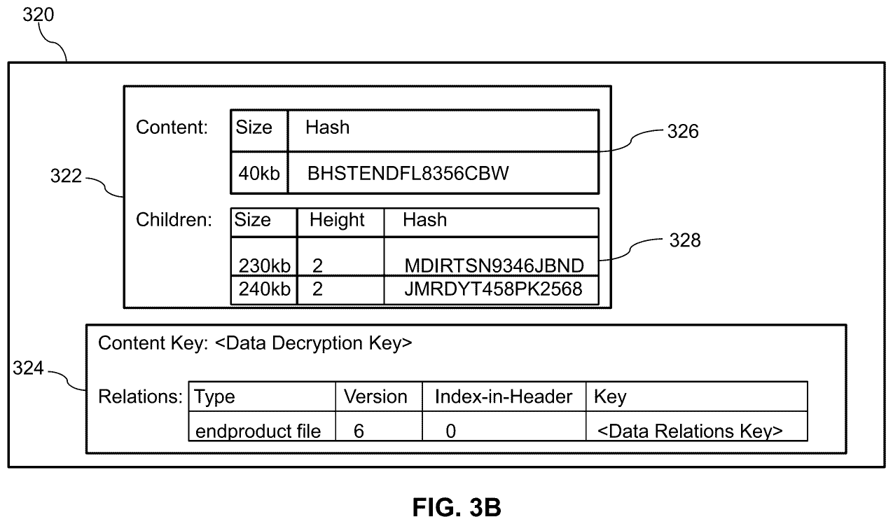

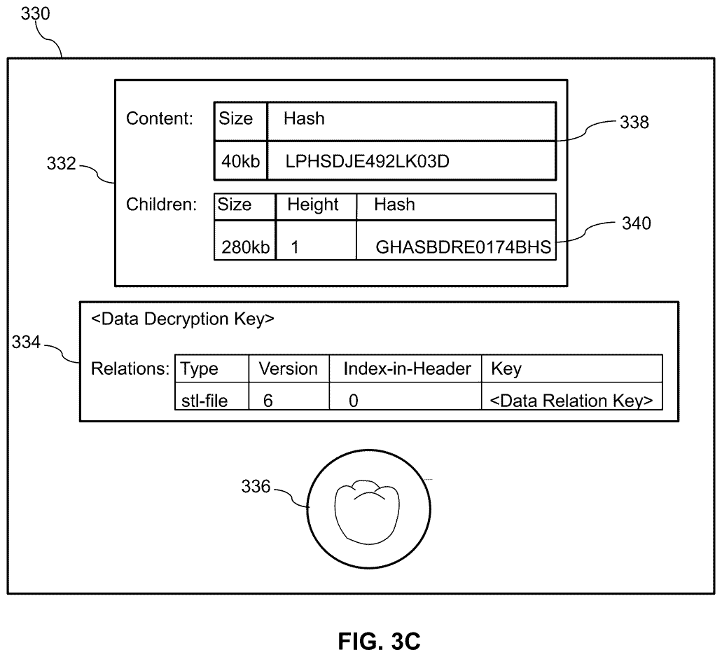

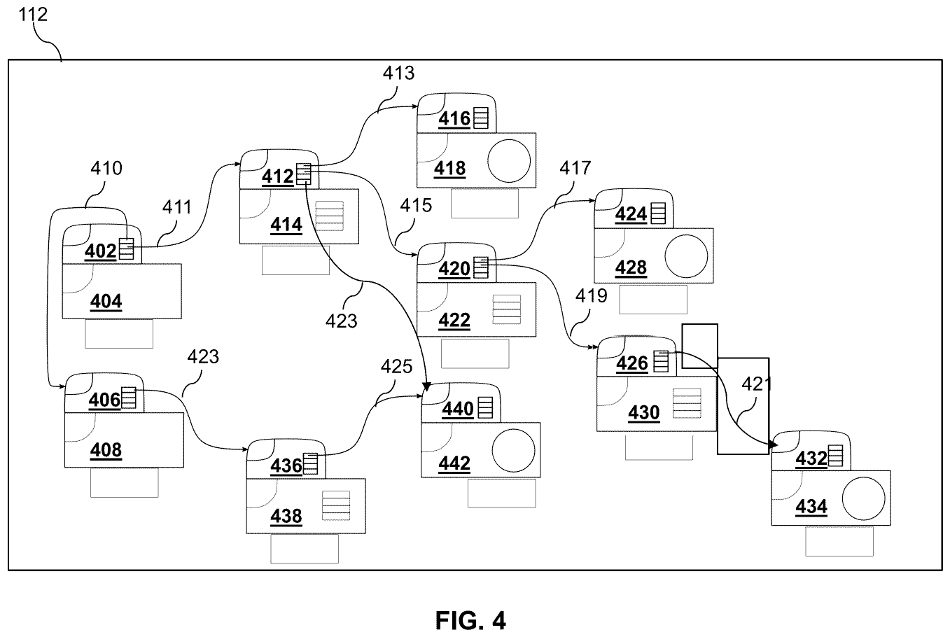

The first node pair includes a first transport header node and a commit node. The first transport header node includes, but is not limited to, a blob reference of the paired commit node. The first node may further include an entity identification of the structured data entity 112, e.g., an alphanumeric string unique to the structured data entity 112. The blob reference of the paired commit node includes, but is not limited to, a size, e.g., in bytes, of the paired commit node, a node height of the paired commit node, and a hash of the paired commit node. The first transport header node may also have a second blob reference including, but not limited to, the size, e.g., in bytes, a node height, and a hash of one or more other transport header nodes referenced by the paired commit node. The commit node includes, but is not limited to, a unique identifier, e.g., an author reference, a digital signature, e.g., the digital signature of the author generated using a private key of a cryptographic key pair, a timestamp, e.g., a date and/or time when the commit node was created, identification of one or more parent commit nodes, e.g., a commit node created after to the current commit node, a root relation node identification, an associated data format version number, e.g., of the data stored in the encrypted data node of the third node pair, and an encrypted data key, e.g., a key for a relation node as described below with reference to FIG. 3B. An example first node pair 302 including an entity identification 303, a first transport header node 304 and a commit node 306 is illustrated in FIG. 3A. The first transport header node 304 includes a commit node blob reference 308 indicating that the paired node, e.g., the commit node 306, is 40 kb in size and has a hash of “XJHS324IUB343IB.” Any suitable hashing algorithm may be used by the system 100 to encrypt and generate hashes of data, e.g., the commit node 306. The first transport header node 304 further includes blob reference 310 identifying two nodes of the structured data entity 112 referenced by the commit node 306, e.g. other transport header nodes, parent commit nodes, etc. The blob reference 310 indicates the first related transport header node is 50 kb in size, has a node height of 4, and a hash of “BHSYE2432NBS352” and the second related transport header node is 50 kb in size, has a node height of 3, and a hash of “NMLOYT275PL2304.” The commit node 306 includes a digital signature, e.g., generated by a private key of a cryptographic key pair associated with an author, a timestamp of 2022-02-01, 10:45 am EST, an indication of no parent commit node, e.g. a value of “0,” an associated data version number of “6.0.,” and a data key “0x16D2B2D.” The data key of the commit node is a relation node key, e.g., a hash of the relation node described below with reference to FIG. 3B, symmetrically encrypted with an entity read-key that is specific to the structured data entity 112. Thus, the data key protects all second and third node pairs from read access from users without the entity read-key. While only a single first node pair is illustrated in FIG. 3A, it can be appreciated that the structured data entity 112 can have multiple first node pairs, e.g., multiple first transport header nodes and multiple commit nodes as discussed in more detail below with reference to FIG. 4.

The second node pair includes a second transport header node and an encrypted relation node. The relation node is encrypted with a symmetric key generated by hashing the relation node. The second transport header node includes, but is not limited to, a blob reference of the paired relation node. The blob reference of the paired relation node includes, but is not limited to, a size, e.g., in bytes, of the paired relation node, and a hash of the paired relation node. The second transport header node may also have a second blob reference including, but not limited to, the size, e.g., in bytes, a node height, and a hash of one or more other transport relation nodes referenced by the associated relation node. The relation node includes, but is not limited to, summary information of data included in an encrypted data node, and a data decryption key for the encrypted data node. The data decryption key is a symmetric key used to encrypt and decrypt the data node of the structured data entity 112. The summary information of data included in an encrypted data node includes, but it not limited to, a data relation type, a data format version, an index-in-header, and a data relations key, etc. The data relation types include, but are not limited to, an entity identification relation, a relation reference, a commit/entity relation, and a binary relation. An entity identification relation is a relation between the encrypted data node and another structured data entity 112, e.g., another structured data entity 112 stored in the database 110. For example, but not limited to, the entity identification relation the alphanumeric entity identification of another structured data entity 112 in the system 100. The entity identification relation is a system-wide unique identifier as it identifies another structured data entity 112 in the database 110. A relation reference is a reference to another encrypted data node of the structured data entity 112 and includes a data decryption key for that encrypted data node. A commit/entity relation is a reference that points to a commit node of another structured data entity 112 stored in the database 110 and includes the data decryption key associated with the top most relation header of that commit node. A binary relation is a direct reference to an encrypted data node. An example second node pair 320 including transport header 322 and an encrypted relation node 324 is illustrated in FIG. 3B. The second transport header node 322 includes a blob reference 326 indicating that the paired relation node, e.g., the relation node 324, is 40 kb in size and has a hash of “BHSTENDFL8356CBW.” Any suitable hashing algorithm may be used by the system 100 to encrypt and generate hashes of data, e.g., the relation node 324. The transport header node 324 further includes blob reference 328 identifying nodes of the structured data entity 112 referenced by the relation node 324, e.g. another relation node. The blob reference 328 indicates the first related transport header is 230 kb in size, has a node height of 2, and a hash of “MDIRTSN9346JBND” and the second related transport header node is 240 kb in size, has a node height of 2, and a hash of “JMRDYT458PK2568.” The relation node 324 includes a data decryption key for the encrypted data node data, a data relation type, e.g., an alphanumeric description of the relation type, and a data format version number of 6, and a data relations key.

The third node pair includes a third transport header node and an encrypted data node. The data node is encrypted with the data encryption key included in the related relation node as described above with reference to FIG. 3B. The data decryption key is a symmetric key generated by hashing the data node. The third transport header node includes, but is not limited to, a blob reference of the associated data node. The blob reference of the associated data node includes, but is not limited to, a size, e.g., in bytes, of the paired data node, and a hash of the paired data node. The third transport header node may also have a second blob reference including, but not limited to, the size, e.g., in bytes, a node height, and a hash of one or more other transport header nodes referenced by the associated data node. The data node includes the encrypted data of the associated structured data entity 112. For example, the encrypted data may be a dental crown design file. An example third node pair 330 including a third transport header 332, an encrypted data node 334, and a data file 336 is illustrated in FIG. 3C. The third transport header 332 includes a blob reference 338 indicating that the associated node, e.g., the data node 334, is 40 kb in size and has a hash of “LPHSDJE492LK03D.” Any suitable hashing algorithm may be used by the system 100 to encrypt and generate hashes of data. The transport header 332 further includes blob reference 340 identifying one other transport headers referenced by the data node 334. The blob reference 340 indicates the related data node is 280 kb in size, has height of 1, and a hash of “GHASBDRE0174BHS.” The data node 334 includes, but is not limited to, a data decryption key for the encrypted data node data, a data relation type, e.g., an alphanumeric description of the relation type, and a data format version number of 6, and a data relations key The data file 336 is a dental crown design file that has been hashed, the resulting hash then used as a symmetric key, e.g., the data decryption key stored in the relation node 324, to encrypt and decrypt the data 336.

While only three node pairs are illustrated, it can be appreciated that a structured data entity 112 can include any number of node pairs greater than three. For example, as illustrated in 4, the structured data entity 112 can include two first node pairs, e.g., first transport header 402 and commit node 404 and first transport header 406 and commit node 408. The first transport header 402 may point to the first transport header 406 in the blob reference of the commit node 404 as illustrated by the line 410. The blob reference of the first transport header 402 may also point the second transport header 412 paired with the relation node 414 as illustrated by the line 411. The blob reference of the second transport node 412 in turn may point to the third transport header 416 paired with data node 418 as illustrated by the line 413. The blob reference of the second transport node 412 may further point to the second transport header 420 paired with relation node 422 as illustrated by the line 415. The blob reference of the second transport header 420 may point to a third transport header 424 paired with a data node 428 as illustrated by the line 417 and a third transport header 426 paired with a relation node 430 as illustrated by the line 419. The blob reference of the third transport header 426 may further point to third transport header 432 paired with data node 434 as illustrated by the line 421. Returning to the first transport header 406, the hash of a blob reference of the first transport header 406 may point to the second transport header 436 paired with the relation node 438 as illustrated by the line 423. The hash of a blob reference of the second transport header 436 may point to the third transport header 440 paired with the data node 442 as illustrated by the line 425. The above connections, e.g., lines 410, 411, 413, 415, 417, 419, 421, 423, and 425, between the node pairs of the structured data entity 112 illustrate two commit nodes, e.g., the commit nodes 402 and 406, pointing to complete versions of data sets, e.g., each commit node points to a history of a single data file. However, the blob reference of a second transport header node paired with an encrypted relation node may point to more than one related third transport header node paired with an encrypted data node. For example, the blob reference of the second transport header 412 may point to third transport header 440 as illustrated by the line 423. In this instance, where a second transport header, e.g., the second transport header 412, points to a third transport header, e.g., the third transport header 440, that stems from a different commit node, e.g., commit node 408 versus commit node 404, means the associated encrypted data, e.g., the data node 442, has been altered in some way at the time both the relation nodes 414 and 438 were created, e.g., two users updated the data of the data node 442 at the same time. As can be appreciated, all the nodes of structured data entity 112 as illustrated in FIG. 4 form a directed acyclic graph (DAG), i.e., the nodes of the structured data entity 112 have a linear ordering that never forms a closed loop. Further, while a single structured data entity 112 is illustrated, two or more structured data entities may reference each other and the data for each referenced structured data entity will automatically be available in the same way as the referring structured data entity's own content. In the system 100, identical data is reused across multiple commit nodes and structured data entities such that the system 100 does not store or transfer the same data more than once.

The transport header nodes of the structured data entity 112 link together the data history associated with the structured data entity 112 such that transfer, indexing, and viewing the data is much more efficient. For example, the root data, e.g., the first data node, is typically small, e.g., a json or .xml file, and larger data like three-dimensional models based on that data are linked from that. This means that to populate indices or projections, e.g., the blob references of the transport header nodes, for search it is sufficient to retrieve just the root data.

Referring back to FIG. 1, the producer devices 102a-102b, the back-up device 104, the proxy device 106, and the storage device 108 may communicate using any network suitable for performing the functions as disclosed herein and may include a local area network (LAN), a wide area network (WAN), a wireless network (e.g., WiFi), a personal area network (PAN) (e.g. Bluetooth), a near-field communication (NFC) network, a mobile communication network, a satellite network, the Internet, fiber optic, coaxial cable, other hardwired networks, infrared, radio frequency (RF), or any combination of the foregoing. Other suitable network types and configurations will be apparent to persons having skill in the relevant art. In general, the network can be any combination of connections and protocols that will support communications between the producer devices 102a-102b, the back-up device 104, the proxy device 106, and the storage device 108. In some embodiments, the network may be optional based on the configuration of the producer devices 102a-102b, the back-up device 104, the proxy device 106, and the storage device 108.

In some embodiments, an optimization is the choice to split up larger files into smaller blobs, which may be known as “chunking.” In order to chunk a large file, the system may divide it into smaller chunks that are stored and encrypted independently. This may be done for any of several reasons, such as to get around potential limitations in the underlying storage system, to better support resumption of large blobs, reduce memory requirements on the client and allow streaming of incomplete data.

In order to reduce the data stored in the system, it is possible to reduce the number of blobs in the DAG without losing any generality or expressiveness. The system may reduce the number of blobs while keeping functionality intact. The purpose of this is to store and send less data, thereby improving the performance and reliability of the system (as there are fewer blobs that can be lost).

To reduce the amount of blobs transferred (as the performance is dependent on not only amount of data, but also the amount of blobs), the system may allow embedding the data directly in the transport headers in certain (or all) cases.

Network mapping allows the application to get the set of all the directly and indirectly connected-to peers, with some information on each peer. This information may include the connectivity information, peer-name, peer-type, untyped errors and/or information on the peers' application name and version. The relevant function may be a OnPremisePeer.NetworkMapping.AllPeersAsync( ). It's recommended that each application calls OnPremisePeer.SetApplicationNameAndVersion(string) with a meaningful application name and version (e.g. Dental Desktop Server 1.18.0.0) during startup. Note that there may be some delay before setting a value on one peer and the result becoming visible on another. The peer type, connection states and the untype errors (e.g., in OtherStatuses) are intended for machine comsumption—the latter being a good candiate for displaying verbatum to the user.

### Producer Device 102a

FIG. 2 illustrates an embodiment of the producer device 102a in the system 100. It will be apparent to persons having skill in the relevant art that the embodiment of the producer device 102a illustrated in FIG. 2 is provided as illustration only and may not be exhaustive to all possible configurations of the producer device 102a suitable for performing the functions as discussed herein. For example, the computer system 600 illustrated in FIG. 6 and discussed in more detail below may be a suitable configuration of the producer device 102. In exemplary embodiments, the producer device 102 in the system 100 produces at least a portion of the dental data or other medical data that is secured and accessed by the system 100.

The producer device 102a may include a receiving device 202. The receiving device 202 may be configured to receive data over one or more networks via one or more network protocols. In some instances, the receiving device 202 may be configured to receive data from the producer device 102b, the back-up device 104, the proxy device 106, the storage device 108, and other systems and entities via one or more communication methods, such as radio frequency, local area networks, wireless area networks, cellular communication networks, Bluetooth, the Internet, etc. In some embodiments, the receiving device 202 may be comprised of multiple devices, such as different receiving devices for receiving data over different networks, such as a first receiving device for receiving data over a local area network and a second receiving device for receiving data via the Internet. The receiving device 202 may receive electronically transmitted data signals, where data may be superimposed or otherwise encoded on the data signal and decoded, parsed, read, or otherwise obtained via receipt of the data signal by the receiving device 202. In some instances, the receiving device 202 may include a parsing module for parsing the received data signal to obtain the data superimposed thereon. For example, the receiving device 202 may include a parser program configured to receive and transform the received data signal into usable input for the functions performed by the processing device to carry out the methods and systems described herein.

The receiving device 202 may be configured to receive data signals electronically transmitted by the producer device 102b, the back-up device 104, the proxy device 106, the storage device 108, that may be superimposed or otherwise encoded with structured data entities, e.g., the structured data entity 112, or a data abstract of a structured data entity, etc. The receiving device 202 may also be configured to receive data signals electronically transmitted by the producer device 102b, the back-up device 104, the proxy device 106, the storage device 108, which may be superimposed or otherwise encoded with data requests, entity key requests, user access key request, etc.

The producer device 102a may also include a communication module 204. The communication module 204 may be configured to transmit data between modules, engines, databases, memories, and other components of the producer device 102a for use in performing the functions discussed herein. The communication module 204 may be comprised of one or more communication types and utilize various communication methods for communications within a computing device. For example, the communication module 204 may be comprised of a bus, contact pin connectors, wires, etc. In some embodiments, the communication module 204 may also be configured to communicate between internal components of the producer device 102a and external components of the producer device 102a, such as externally connected databases, display devices, input devices, etc. The producer device 102a may also include a processing server. The processing device may be configured to perform the functions of the producer device 102a discussed herein as will be apparent to persons having skill in the relevant art. In some embodiments, the processing server may include and/or be comprised of a plurality of engines and/or modules specially configured to perform one or more functions of the producer device 102a, such as a querying module 214, generation module 216, validation module 218, etc. As used herein, the term “module” may be software or hardware particularly programmed to receive an input, perform one or more processes using the input, and provides an output. The input, output, and processes performed by various modules will be apparent to one skilled in the art based upon the present disclosure.

The producer device 102a may also include a memory 206. The memory 206 may be configured to store data for use by the producer device 102a in performing the functions discussed herein, such as public and private keys, symmetric keys, etc. The memory 206 may be configured to store data using suitable data formatting methods and schema and may be any suitable type of memory, such as read-only memory, random access memory, etc. The memory 206 may include, for example, encryption keys and algorithms, communication protocols and standards, data formatting standards and protocols, program code for modules and application programs of the processing device, and other data that may be suitable for use by the producer device 102a in the performance of the functions disclosed herein as will be apparent to persons having skill in the relevant art. In some embodiments, the memory 206 may be comprised of or may otherwise include a relational database that utilizes structured query language for the storage, identification, modifying, updating, accessing, etc. of structured data sets stored therein. The memory 206 may be configured to store, for example, the data abstract 208 of the database 110, the entity key 210, the user access key 212, communication information for the producer device 102b, the back-up device 104, the proxy device 106, the storage device 108, address generation and validation algorithms, digital signature generation and validation algorithms, hashing algorithms for generating reference values, e.g., transport header node values, rules regarding generation of new nodes in the structured data entity 112, rules regarding generation of new structured data entities in the database 110, rules regarding structured data entity access, etc.

The data abstract 208 includes the transport headers (e.g., the first transport header node 304, the second transport header node 322, and the third transport header node 332 of FIGS. 3A-3C, and/or the transport headers 402, 406, 412, 416, 420, 424, 426, 432, 436, and 440 of FIG. 4) of the structured data entities of the database 110. The data abstract 208 gives the devices in the system 100 knowledge of the data context (e.g. the unencrypted transport header nodes of the structured data entities 112) of the database 110 without actually having access to the data. For example, the Data Abstract 208 enables the devices of the system 100 to traverse the transport header nodes of the database 110 without the ability to decrypt the encrypted parts of the database 110 (e.g., the encrypted relation nodes and the encrypted data nodes). The entity key 210 is a decryption key for decrypting the data key stored in the one or more commit nodes of the structured data entity 112. For example, the entity key 210 is a public key of a cryptographic key pair associated with the structured data entity 112. The user access key 212 is associated with one or more users of the devices of the system 100 and grants the associated user access to one or more entity keys 210. For example, the database 110 may include structured data entities 112a-d and a user associated with the producer device 102a may have an access key 212 that grants that user access to entity keys 210a-b for the structured data entities 112a-b, but does not grant that user access to the entity keys 210c-d for the structured data entities 112c-d. The user access key 212 may be a group access key that grants a group of one or more users access to one or more entity keys 210 associated with the user access key 212. For example, the users of the system 100 may be broken down into three groups and each group would have its own access key 212 that grants each user of that group access to a unique subset of the one or more entity keys 210.

The processing device 102 may include a querying module 214. The querying module 214 may be configured to execute queries on databases to identify information. The querying module 214 may receive one or more data values or query strings, and may execute a query string based thereon on an indicated database, such as the memory 206 of the producer device 102a or the database 110 of the storage device 108 to identify information stored therein. The querying module 214 may then output the identified information to an appropriate engine or module of the producer device 102a as necessary. The querying module 214 may, for example, execute a query on the memory 206 to access the data abstract 208, the entity key 210, and/or the user access key 212. Further, the querying module 214 may, for example, execute a query on the database 110 for one or more structured data entities 112 and the data associated therewith.

The processing device 102 may also include a generation module 216. The generation module 216 may be configured to generate data for use by the producer device 102a in performing the functions discussed herein. The generation module 216 may receive instructions as input, may generate data based on the instructions, and may output the generated data to one or more modules of the producer device 102a. For example, the generation module 216 may be configured to generate one or more structured data entities 112 and/or update one or more structured data entities 112 including generating one or more transport header nodes, one or more commit nodes, one or more relation nodes, and/or one or more data nodes, etc. The nodes of the structured data entities 112 themselves are immutable and any updates to the structured data entities 112 results in the generation of a new node. Further the generation module 216 may be configured to generate one or more hashes such as, but not limited to, hashes of the data nodes, hashes of the relation nodes, and hashes of the commit nodes of the structured data entity 112.

The producer device 102a may also include a validation module 218. The validation module 218 may be configured to perform validations for the producer device 102a as part of the functions discussed herein. The validation module 218 may receive instructions as input, which may also include data to be used in performing a validation, may perform a validation as requested, and may output a result of the validation to another module or engine of the producer device 102a. The validation module 218 may, for example, be configured to validate one or more cryptographic signatures and/or one or more hashes includes in the structured data entities 112.

The producer device 102a may also include a transmitting device 220. The transmitting device 220 may be configured to transmit data over one or more networks via one or more network protocols. In some instances, the transmitting device 220 may be configured to transmit data to the producer device 102b, the back-up device 104, the proxy device 106, the storage device 108, and other entities via one or more communication methods, local area networks, wireless area networks, cellular communication, Bluetooth, radio frequency, the Internet, etc. In some embodiments, the transmitting device 220 may be comprised of multiple devices, such as different transmitting devices for transmitting data over different networks, such as a first transmitting device for transmitting data over a local area network and a second transmitting device for transmitting data via the Internet. The transmitting device 220 may electronically transmit data signals that have data superimposed that may be parsed by a receiving computing device. In some instances, the transmitting device 220 may include one or more modules for superimposing, encoding, or otherwise formatting data into data signals suitable for transmission.

The transmitting device 220 be configured to electronically transmit data signals to the producer device 102b, the back-up device 104, the proxy device 106, and the storage device 108 that are superimposed or otherwise encoded with requests for data, e.g., the encrypted data nodes of structured data entity 112, stored in the database 110. The transmitting device 220 may also be configured to electronically transmit data signals to the producer device 102b, the back-up device 104, the proxy device 106, the storage device 108, which may be superimposed or otherwise encoded with, new structured data entities generated by the producer device 102a, updated structured data entities 112, etc.

### Exemplary Method for Distributed Storage and Management of Data

FIG. 5 illustrates a method 500 for distributed storage and management of data in accordance with exemplary embodiments.

The method 500 can include block 502 of storing, by a computing device in a network (e.g., one of the producer devices 102a-102b), a data abstract (e.g., the data abstract 208) interfaced with a database (e.g., the database 110). The data abstract stores one or more structured data entities (e.g., the structured data entity 112). Each of the one or more structured data entities includes at least three node pairs. The first node pair includes a first transport header node (e.g., the first transport header node 304) paired with a commit node (e.g., the commit node 306). The first transport header includes at least a blob reference of the commit node (e.g., the blob reference of the commit node 308). The first transport header includes, but is not limited to, a hash of the commit node (e.g., the commit node 306) and a hash of a second transport header node (e.g., the second transport header node 322). The commit node includes, but is not limited to, a unique identifier, a digital signature, and a data key. The data key is a relation node key encrypted with an entity key specific to an associated structured data entity. The second node pair includes a second transport header node (e.g., the second transport header node 322) paired with an encrypted relation node (e.g., the relation node 324) encrypted with the relation node key, e.g., a symmetric key generated by hashing the relation node. The second transport header node includes, but is not limited to, a blob reference 326 of the encrypted relation node (e.g., the relation node). The second transport header node includes a hash of the encrypted relation node and another blob reference 328 includes a hash of a third transport header node (e.g. the third transport header node 332). The encrypted relation node includes, but is not limited to, a summary of data included in an encrypted data node and a data decryption key for the encrypted data node. The third node pair includes a third transport header node (e.g. the third transport header node 332) paired with an encrypted data node (e.g., the data node 334), and a data file (e.g., the data file 336). The third transport header node includes blob reference which includes, but is not limited to, a size of the encrypted data node and a hash of the encrypted data node (e.g., the data node 334). The encrypted data node includes, but is not limited to, encrypted data related to the associated structured data entity 112 encrypted using the data encryption key. The producer device 102a may be part of a network of devices (e.g., the producer device 102b, the back-up device 104, the proxy device 106, and the storage node 108). Each of the devices of the network is usually associated with one or more users and the one or more users are defined as one or more of: a producer peer, a proxy peer, or a backup peer; and/or a storage peer. Further, each user associated with the devices is assigned and/or associated with a user access key (e.g., the user access key 212). The user access key grants its associated user permissions to access one or more entity keys 210 associated with one or more structured data entities 112. For example, the database 110 may include structured data entities 112a-d and a user associated with the producer device 102a may have an access key 212 that grants that user access to entity keys 210a-b for the structured data entities 112a-b, but does not grant that user access to the entity keys 210c-d for the structured data entities 112c-d. The user access key 212 may be a group access key that grants a group of one or more users access to one or more entity keys 210 associated with the user access key 212. For example, the users of the system 100 may be broken down into three groups and each group would have its own access key 212 that grants each user of that group access to a unique subset of the one or more entity keys 210. In this way all the users of the system 100 have knowledge of the data (e.g., the structured data entity 112) stored in the database 110, e.g., the users know the data is there based on the unencrypted transport headers of the structured data entity 112 (e.g., the data abstract 208). However, only users who have a user access key that grants them permission to one or more entity keys, will actually be able to access the data stored in the one or more structured data entities 112. Further, the setup of the system 100 allows for a more efficient access to stored data (e.g., the database 110), by providing all devices in the system 100 with the data abstract 208, e.g., the one or more transport headers of the structured data entity 112, which is of a considerably smaller storage size then the entire database 110. Thus, in the system 100, every device (e.g., the producer devices 102a-102b, the back-up device, and the proxy device 106) does not need to store the entire database 110 or request all the data stored in the database 110 in order to know what data is stored within the database 110, but rather can use the data abstract to find exactly what data they need and only that data. In an exemplary embodiment, the producer devices 102a-102b are dental devices that generate medical data, e.g., dental data, and the storage device 108 is a medical office storage device, e.g. a dental office storage device.

The method 500 can include block 504 of transmitting, by the computing device (e.g., one of the producer devices 102a-102b), a request for the first transport header node (e.g., the first transport header node 304) and the paired commit node (e.g., the commit node 306) of a specific structured data entity (e.g., the structured data entity 112) of the one or more structured data entities

The method 500 can include block 506 of receiving, by the computing device, the first transport header node and the paired commit node of the specific structured data entity (e.g., the structured data entity 112).

The method 500 can include block 508 of storing, by the computing device (e.g., one of the producer devices 102a-102b), a user access key (e.g., the user access key 212) in a memory (e.g. the memory 206) of the computing device. While the user access is illustrated as being stored within the computing device, it can be appreciated that the user access key may be stored in a separate computing device, e.g., the storage device 108, or another access management computing device. The user access key grants permission, e.g., to a user of the producer device 102a, to access one or more of a plurality of entity keys. Each of the plurality of entity keys associated with a structured data entity (e.g., the structured data entity 112) of the one or more structured data entities.

The method 500 can include block 510 of identifying, by the computing device (e.g., one of the producer devices 102a-102b), a specific entity key (e.g., the entity key 210) of the one or more entity keys associated with the specific structured data entity (e.g., the structured data entity 112). For example, a user of the computing device (e.g., one of the producer devices 102a-102b), may be associated with an entity key 212a, that grants access to entity key 210 a, but not any other entity keys 210, e.g., entity keys 210b-n.

The method 500 can include block 512 of decrypting, by the computing device (e.g., one of the producer devices 102a-102b), the data key using the specific entity key (e.g., the entity key 210).

The method 500 can include block 514 of identifying, by the computing device (e.g., one of the producer devices 102a-102b), the second transport header node (e.g. the second transport header node 322) and the paired encrypted relation node (e.g., the relation node 324) based on the commit node blob reference (e.g. the commit node blob reference 308) of the received commit node (e.g. the commit node 306).

The method 500 can include block 516 of decrypting, by the computing device (e.g., one of the producer devices 102a-102b), the encrypted relation node (e.g. the relation node 324) using the relation node key of the decrypted data key and a block 518 of identifying, by the computing device, the third transport header node (e.g. the third transport header node 332) and the paired encrypted data node (e.g. the data node 334) based on the encrypted relation node.

The method 500 can include block 520 of decrypting, by the computing device (e.g., one of the producer devices 102a-102b), the encrypted data of the encrypted data node (e.g. the data node 334) using the data decryption key of the decrypted relation node (e.g. the relation node 324) and the entity key (e.g. the entity key 210) specific to the associated structured data entity (e.g. the structured data entity 112). The method 500 may proceed to either the block 522 and/or the block 538 from the block 520. In the blocks 522-536, the computing device (e.g., one of the producer devices 102a-102b), generates a data update to data stored in an existing structured data entity (e.g., the structured data entity 112). In the block 538, the computing device (e.g., one of the producer devices 102a-102b), generates a new structured data entity to be added to the database 110.

The method 500 can include block 522 of generating, by the computing device (e.g., one of the producer devices 102a-102b), a data update of encrypted data of a specific encrypted data node of a specific structured data entity of the one or more structured data entities 112. The nodes of the structured data entities 112 are immutable and thus any updates to the data associated with the structured data entities results in a new node. The computing device may generate a new third transport header node paired with a new data node, the new third transport header node including at least a size of the new data node and a hash of the new data node at block 324.

The method 500 can include block 526 of generating, by the computing device (e.g., one of the producer devices 102a-102b), a new data decryption key, the new data decryption key generated by hashing the new data node.

The method 500 can include block 527 of encrypting, by the computing device (e.g., one of the producer devices 102a-102b), the new data node with the new data encryption key.

The method 500 can include a block 528 of generating, by the computing device (e.g., one of the producer devices 102a-102b), a new second transport header and a new relation node pair based on the new third transport header node and a new data node. One or more blob references of the new relation node includes at least a hash of the new third transport header node and a hash of the new relation node, the new relation node including a summary of the data update included in the encrypted new data node and the new data decryption key.

The method 500 can include block 530 of generating, by the computing device (e.g., one of the producer devices 102a-102b), a new relation node key. The new relation node key generated by hashing the new relation node.

The method 500 can include block 532 of encrypting, by the computing device (e.g., one of the producer devices 102a-102b), the new relation node with the new relation node key.

The method 500 can include block 534 of generating, by the computing device (e.g., one of the producer devices 102a-102b), a new first transport header node and a new commit node pair based on the data update, wherein one or more blob references of the commit node in the new first transport header node includes at least a hash of at least one previously generated commit node (e.g., the commit node 306) of the associated structured data entity (e.g. the structured data entity 112). The new commit node includes a new data key, the new data key being the new relation node key encrypted with the entity key of the specific structured data entity.

The method 500 can include block 536 of transmitting, by the computing device (e.g., one of the producer devices 102a-102b), the new commit node of the specific structured data entity of the one or more structured data entities to one or more second computing devices in the network (e.g., the storage device 108). The storage device 108 adds the new commit node to the database 110.

The method 500 can include block 538 of generating, by the computing device (e.g., one of the producer devices 102a-102b) a new structured data entity data to be added to the database (e.g. the database 110). The new structured data entity can include data identifying an existing structured data entity (e.g. the structured data entity 112) to which the new structured data entity is to be associated. The new structured data entity includes at least, a new first transport header node and new commit node pair, a new second transport header node and new encrypted relation node pair, and a new third transport header node and new encrypted data node pair. Generating the new structured data entity includes linking, by the computing device (e.g., one of the producer devices 102a-102b), the new structured data entity with the existing structured data entity (e.g. the structured data entity 112). Further, linking the new structured data entity with the existing structured data entity includes inserting a hash of the encrypted relation node of the existing structured data entity (e.g., the encrypted relation node 324) in a blob reference of the new first transport header node. Generating the new structured data entity further includes transmitting, by the computing device (e.g., one of the producer devices 102a-102b), the new structured data entity to one or more second computing devices in the network (e.g., the back-up device 104, the proxy device 106, and/or the storage device 108).

Soft-locks may provide a mechanism to improve collaboration in a distributed system by allowing the applications to express that certain workflows or data sets are in use. This enables the application to prevent multiple users from working on the same data at the same time. There is no built-in link between entities and soft-locks, instead may be entirely up to the application to decide how to interpret soft-locks. The soft-locks may be propagated in much the same way as entities this also implies that soft-locks can only be propagated if there is in fact a connection between peers. Peers that are not connected won't be able to see lock belonging to the other. They may instead be able to make concurrent changes to the data that would have been protected by the locks (typically entities).

### Computer System Architecture

FIG. 6 illustrates a computer system 600 in which embodiments of the present disclosure, or portions thereof, may be implemented as computer-readable code. For example, the producer devices 102a-102b, the back-up device 104, the proxy device 106, and/or the storage device 108 of FIG. 1 may be implemented in the computer system 600 using hardware, software, firmware, non-transitory computer readable media having instructions stored thereon, or a combination thereof and may be implemented in one or more computer systems or other processing systems. Hardware, software, or any combination thereof may embody modules and components used to implement the method of FIG. 5.

If programmable logic is used, such logic may execute on a commercially available processing platform configured by executable software code to become a specific purpose computer or a special purpose device (e.g., programmable logic array, application-specific integrated circuit, etc.). A person having ordinary skill in the art may appreciate that embodiments of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device. For instance, at least one processor device and a memory may be used to implement the above described embodiments.

A processor unit or device as discussed herein may be a single processor, a plurality of processors, or combinations thereof. Processor devices may have one or more processor “cores.” The terms “computer program medium,” “non-transitory computer readable medium,” and “computer usable medium” as discussed herein are used to generally refer to tangible media such as a removable storage unit 618, a removable storage unit 622, and a hard disk installed in hard disk drive 612.

Various embodiments of the present disclosure are described in terms of this example computer system 600. After reading this description, it will become apparent to a person skilled in the relevant art how to implement the present disclosure using other computer systems and/or computer architectures. Although operations may be described as a sequential process, some of the operations may in fact be performed in parallel, concurrently, and/or in a distributed environment, and with program code stored locally or remotely for access by single or multiprocessor machines. In addition, in some embodiments the order of operations may be rearranged without departing from the spirit of the disclosed subject matter.

Processor device 604 may be a special purpose or a general purpose processor device specifically configured to perform the functions discussed herein. The processor device 604 may be connected to a communications infrastructure 606, such as a bus, message queue, network, multi-core message-passing scheme, etc. The network may be any network suitable for performing the functions as disclosed herein and may include a local area network (LAN), a wide area network (WAN), a wireless network (e.g., WiFi), a mobile communication network, a satellite network, the Internet, fiber optic, coaxial cable, infrared, radio frequency (RF), or any combination thereof. Other suitable network types and configurations will be apparent to persons having skill in the relevant art. The computer system 600 may also include a main memory 608 (e.g., random access memory, read-only memory, etc.), and may also include a secondary memory 610. The secondary memory 610 may include the hard disk drive 612 and a removable storage drive 614, such as a floppy disk drive, a magnetic tape drive, an optical disk drive, a flash memory, etc.

The removable storage drive 614 may read from and/or write to the removable storage unit 618 in a well-known manner. The removable storage unit 618 may include a removable storage media that may be read by and written to by the removable storage drive 614. For example, if the removable storage drive 614 is a floppy disk drive or universal serial bus port, the removable storage unit 618 may be a floppy disk or portable flash drive, respectively. In one embodiment, the removable storage unit 618 may be non-transitory computer readable recording media.

In some embodiments, the secondary memory 610 may include alternative means for allowing computer programs or other instructions to be loaded into the computer system 600, for example, the removable storage unit 622 and an interface 620. Examples of such means may include a program cartridge and cartridge interface (e.g., as found in video game systems), a removable memory chip (e.g., EEPROM, PROM, etc.) and associated socket, and other removable storage units 622 and interfaces 620 as will be apparent to persons having skill in the relevant art.

Data stored in the computer system 600 (e.g., in the main memory 608 and/or the secondary memory 610) may be stored on any type of suitable computer readable media, such as optical storage (e.g., a compact disc, digital versatile disc, Blu-ray disc, etc.) or magnetic tape storage (e.g., a hard disk drive). The data may be configured in any type of suitable database configuration, such as a relational database, a structured query language (SQL) database, a distributed database, an object database, etc. Suitable configurations and storage types will be apparent to persons having skill in the relevant art.

The computer system 600 may also include a communications interface 624. The communications interface 624 may be configured to allow software and data to be transferred between the computer system 600 and external devices. Exemplary communications interfaces 624 may include a modem, a network interface (e.g., an Ethernet card), a communications port, a PCMCIA slot and card, etc. Software and data transferred via the communications interface 624 may be in the form of signals, which may be electronic, electromagnetic, optical, or other signals as will be apparent to persons having skill in the relevant art. The signals may travel via a communications path 626, which may be configured to carry the signals and may be implemented using wire, cable, fiber optics, a phone line, a cellular phone link, a radio frequency link, etc.

The computer system 600 may further include a display interface 602. The display interface 602 may be configured to allow data to be transferred between the computer system 600 and external display 630. Exemplary display interfaces 602 may include high-definition multimedia interface (HDMI), digital visual interface (DVI), video graphics array (VGA), etc. The display 630 may be any suitable type of display for displaying data transmitted via the display interface 602 of the computer system 600, including a cathode ray tube (CRT) display, liquid crystal display (LCD), light-emitting diode (LED) display, capacitive touch display, thin-film transistor (TFT) display, etc.

Computer program medium and computer usable medium may refer to memories, such as the main memory 608 and secondary memory 610, which may be memory semiconductors (e.g., DRAMs, etc.). These computer program products may be means for providing software to the computer system 600. Computer programs (e.g., computer control logic) may be stored in the main memory 608 and/or the secondary memory 610. Computer programs may also be received via the communications interface 624. Such computer programs, when executed, may enable computer system 600 to implement the present methods as discussed herein. In particular, the computer programs, when executed, may enable processor device 604 to implement the processes and methods illustrated by FIG. 5, as discussed herein. Accordingly, such computer programs may represent controllers of the computer system 600. Where the present disclosure is implemented using software, the software may be stored in a computer program product and loaded into the computer system 600 using the removable storage drive 614, interface 620, and hard disk drive 612, or communications interface 624.

The processor device 604 may comprise one or more modules or engines configured to perform the functions of the computer system 600. Each of the modules or engines may be implemented using hardware and, in some instances, may also utilize software, such as corresponding to program code and/or programs stored in the main memory 608 or secondary memory 610. In such instances, program code may be compiled by the processor device 604 (e.g., by a compiling module or engine) prior to execution by the hardware of the computer system 600. For example, the program code may be source code written in a programming language that is translated into a lower level language, such as assembly language or machine code, for execution by the processor device 604 and/or any additional hardware components of the computer system 600. The process of compiling may include the use of lexical analysis, preprocessing, parsing, semantic analysis, syntax-directed translation, code generation, code optimization, and any other techniques that may be suitable for translation of program code into a lower level language suitable for controlling the computer system 600 to perform the functions disclosed herein. It will be apparent to persons having skill in the relevant art that such processes result in the computer system 600 being a specially configured computer system 600 uniquely programmed to perform the functions discussed above.

Techniques consistent with the present disclosure provide, among other features, systems and methods for distributed storage and management of data. While various exemplary embodiments of the disclosed system and method have been described above it should be understood that they have been presented for purposes of example only, not limitations. It is not exhaustive and does not limit the disclosure to the precise form disclosed. Modifications and variations are possible in light of the above teachings or may be acquired from practicing of the disclosure, without departing from the breadth or scope. Although operations can be described as a sequential process, some of the operations can in fact be performed in parallel, concurrently, and/or in a distributed environment, and with program code stored locally or remotely for access by single or multi-processor machines. In addition, in some embodiments the order of operations can be rearranged without departing from the spirit of the disclosed subject matter. It will be appreciated by those skilled in the art that the present disclosure can be embodied in other specific forms without departing from the spirit or essential characteristics thereof. The presently disclosed embodiments are therefore considered in all respects to be illustrative and not restrictive. The scope of the disclosure is indicated by the appended claims rather than the foregoing description, and all changes that come within the meaning, range, and equivalence thereof are intended to be embraced therein.

### List of Items

1. A method for distributed storage and management of data, the method comprising:
   storing, by a computing device in a network, a data abstract interfaced with a database, data abstract storing one or more structured data entities, each of the one or more structured data entities including at least three node pairs including:
      a first transport header node paired with a commit node, the first transport header node including at least a blob reference of the commit node, one of the blob references of the commit node including a hash of the commit node and size of the one of the blob references of the commit node and a second blob reference of the first transport header including a hash of a second transport header node and size of the second blob reference of the first transport header, the commit node including a unique identifier, a digital signature, and a data key, the data key being a relation node key encrypted with an entity key specific to an associated structured data entity of the one or more structured data entities;
      the second transport header node paired with an encrypted relation node encrypted with the relation node key, the second transport header including at least a blob reference of the encrypted relation node, one of the blob references of the encrypted relation node including a hash of a third transport header node and size of the one of the blob references of the encrypted relation node and a second blob reference of the second transport header including a hash of the encrypted relation node and size of the second blob reference of the second transport header, the encrypted relation node including a summary of data included in an encrypted data node and a data decryption key for the encrypted data node;
      the third transport header node paired with the encrypted data node and an encrypted data file, the third transport header node including a blob reference of the data node, the blob reference of the data node including at least a size of the encrypted data node and a hash of the encrypted data node, wherein the encrypted data node includes encrypted data related to the associated structured data entity encrypted using the data encryption key;
   transmitting, by the computing device, a request for the first transport header node and paired commit node of a specific structured data entity of the one or more structured data entities; and
   receiving, by the computing device, the first transport header node and the paired commit node of the specific structured data entity.
2. The method of item 1, comprising:
   storing, by the computing device, a user access key, the user access key granting permission to access one or more of a plurality of entity keys, each of the plurality of entity keys associated with a structured data entity of the one or more structured data entities;
   identifying, by the computing device, a specific entity key of the one or more entity keys associated with the specific structured data entity;
   decrypting, by the computing device, the data key using the specific entity key;
   identifying, by the computing device, the second transport header node and the paired encrypted relation node based on the blob reference of the received commit node in the first transport header node;
   decrypting, by the computing device, the encrypted relation node using the relation node key of the decrypted data key;
   identifying, by the computing device, the third transport header node and the paired encrypted data node based on the encrypted relation node; and
   decrypting, by the computing device, the encrypted data of the encrypted data node using the data decryption key of the decrypted relation node.
3. The method of any of the preceding items 1 to 2, wherein the network includes one or more computing devices associated with one or more users, the one or more computing devices defined as at least one of: a storage peer, a producer peer, a proxy peer, or a backup peer; and
   wherein the one or more users form one or more groups of users, each group of users associated with a user access key.
4. The method of any of the preceding items 1 to 3, wherein the blob reference of the commit node in the first transport header node includes a hash of the commit node and the second blob reference in the first transport header node comprises at least one hash of at least one a second transport header node of the associated structured data entity.
5. The method of any of the preceding items 1 to 4, wherein the encrypted relation node blob reference in the second transport header node includes a hash of the relation node and the second blob reference in the second transport header node comprises at least one hash of at least one third transport header node of the associated structured data entity.
6. The method of any of the preceding items 1 to 5, wherein the database is cloud storage.
7. The method of any of the preceding items 1 to 6, wherein the network is a distributed network.
8. The method of any of the preceding items 1 to 7, wherein the encrypted data is medical patient data.
9. The method of any of the preceding items 1 to 8, wherein the node pairs of each of the one or more structured data entities form a directed acyclic graph.
10. The method of any of the preceding items 1 to 9, comprising:
   generating, by the computing device, a data update of encrypted data of a specific encrypted data node of a specific structured data entity of the one or more structured data entities;
   generating, by the computing device, a new third transport header node paired with a new data node, the new third transport header node including at least a size of the new data node and a blob reference of the new data node;
   generating, by the computing device, a new data decryption key, the new data decryption key generated by hashing the new data node;
   encrypting, by the computing device, the new data node with the new data encryption key;
   generating, by the computing device, a new second transport header and a new relation node pair based on the new third transport header node and a new data node, wherein one or more blob references of the new relation node includes at least a hash of the new third transport header node and a hash of the new relation node, the new relation node including a summary of the data update included in the encrypted new data node and the new data decryption key;
   generating, by the computing device a new relation node key, the new relation node key generated by hashing the new relation node;
   encrypting, by the computing device, the new relation node with the new relation node key;
   generating, by the computing device, a new first transport header node and a new commit node pair based on the data update, wherein one or more blob references of the commit node in the new first transport header node includes at least a hash of at least one previously generated commit node of the associated structured data entity, and the new commit node includes a new data key, the new data key being the new relation node key encrypted with the entity key of the specific structured data entity; and
   transmitting, by the computing device, the new first transport header and commit node pair of the specific structured data entity of the one or more structured data entities to one or more second computing devices in the network.
11. The method of any of the preceding items 1 to 10, comprising:
   generating, by the computing device, a new structured data entity data to be added to the database, the new structured data entity including data identifying an existing structured data entity of the one or more structured data entities to which the new structured data entity is to be associated, wherein the generating of the new structured data entity includes:
      generating, by the computing device in the network, a new first transport header node and new commit node pair, a new second transport header node and new encrypted relation node pair, and a new third transport header node and new encrypted data node pair;
      linking, by the computing device, the new structured data entity with the existing structured data entity; and
      transmitting, by the computing device, the new structured data entity to one or more second computing devices in the network.
12. The method of any of the preceding items 1 to 11, wherein the new commit node includes the unique identifier of the existing structured data entity; and
   wherein the linking includes:
      inserting a hash of the encrypted relation node of the existing structured data entity in a blob reference of the new first transport header node.
13. The method of any of the preceding items 1 to 12, wherein each of the one or more structured data entities are associated with a dental patient.
14. The method of any of the preceding items 1 to 13, wherein the storage peer is associated with a dental office, and the producer peer is a computing device that generates dental data.
15. The method of any of the preceding items 1 to 14, wherein the encrypted data of the one or more structured data entities is stored encrypted in the database such that the user of the database is permitted to only read the transport header nodes of the node pairs and the commit nodes without one or more decryption keys.
16. A system for distributed storage and management of data, the system comprising:
   a memory of a processor in a network configured to:
      store a data abstract interfaced with a database, the data abstract storing the one or more structured data entities, each of one or more structured data entities including at least three node pairs including:
      a first transport header node paired with a commit node, the first transport header node including at least a blob reference of the commit node, one of the blob references of the commit node including a hash of the commit node and size of the one of the blob references of the commit node and a second blob reference of the first transport header including a hash of a second transport header node and size of the second blob reference of the first transport header, the commit node including a unique identifier, a digital signature, and a data key, the data key being a relation node key encrypted with an entity key specific to an associated structured data entity of the one or more structured data entities;
      the second transport header node paired with an encrypted relation node encrypted with the relation node key, the second transport header including at least a blob reference of the encrypted relation node, one of the blob references of the encrypted relation node including a hash of a third transport header node and size of the one of the blob references of the encrypted relation node and a second blob reference of the second transport header including a hash of the encrypted relation node and size of the second blob reference of the second transport header, the encrypted relation node including a summary of data included in an encrypted data node and a data decryption key for the encrypted data node;
      the third transport header node paired with the encrypted data node and an encrypted data file, the third transport header node including a blob reference of the data node, the blob reference of the data node including at least a size of the encrypted data node and a hash of the encrypted data node, wherein the encrypted data node includes encrypted data related to the associated structured data entity encrypted using the data encryption key;
   a transmitting device of the processor configured to transmit a request for the first transport header node and the paired commit node of a specific structured data entity of the one or more structured data entities; and
   a receiving device of the processor configured to receive the first transport header node
   and the paired commit node of the specific structured data entity.
17. The system of claim 16, further comprising:
   the memory of the processor configured to:
      store a user access key granting permission to access one or more of a plurality of entity keys, each of the plurality of entity keys associated with a specific structured data entity of the one or more structured data entities;
   a processing device of the processor configured to:
      identify a specific entity key of the one or more entity keys associated with the specific structured data entity;
      decrypt the data key using the specific entity key;
      identify the second transport header node and the paired encrypted relation node based on the blob reference of the received commit node in the first transport header node;
      decrypt the encrypted relation node using the relation node key of the decrypted data key;
      identify the third transport header node and the paired encrypted data node based on the encrypted relation node; and
      decrypt the encrypted data of the encrypted data node using the data decryption key of the decrypted relation node.
18. The system of any of the preceding items 16 to 17, wherein the network includes one or more computing devices associated with one or more users, the one or more computing devices defined as at least one of: a storage peer, a producer peer, a proxy peer, or a backup peer; and
   wherein the one or more users form one or more groups of users, each group of users associated with a user access key.
19. The system of any of the preceding items 16 to 18, wherein the blob reference of the commit node in the first transport header node includes a hash of the commit node and the second blob reference in the first transport header node comprises at least one hash of at least one a second transport header node of the associated structured data entity.
20. The system of any of the preceding items 16 to 19, wherein the blob reference in the second transport header node includes a hash of a plurality of the relation node and the second blob reference in the second transport header node comprises at least one hash of at least one third transport header node of the associated structured data entity.
21. The system of any of the preceding items 16 to 20, wherein the database is cloud storage
22. The system of any of the preceding items 16 to 21, wherein the network is a distributed network.
23. The system of any of the preceding items 16 to 22, wherein the encrypted data is medical patient data.
24. The system of any of the preceding items 16 to 23, wherein the node pairs of each of the one more structured data entities form a directed acyclic graph.
25. The system of any of the preceding items 16 to 24, comprising:
   the processing device of the processor configured to:
      generate a data update of encrypted data of a specific encrypted data node of a specific structured data entity of the one or more structured data entities;
      generate a new third transport header node paired with a new data node, the new third transport header node including at least a size of the new data node and a blob reference of the new data node;
      generate a new data decryption key, the new data decryption key generated by hashing the new data node;
      encrypt the new data node with the new data encryption key;
      generate a new second transport header and a new relation node pair based on the new third transport header node and a new data node, wherein one or more blob references of the new relation node includes at least a hash of the new third transport header node and a hash of the new relation node, the new relation node including a summary of the data update included in the encrypted new data node and the new data decryption key;
      generate the new relation node key generated by hashing the new relation node;
      encrypt the new relation node with the new relation node key;
      generate a new first transport header node and a new commit node pair based on the data update, wherein one or more blob references of the commit node in the new first transport header node includes at least a hash of at least one previously generated commit node of the associated structured data entity, wherein the new commit node includes a new data key, the new data key being the new relation node key encrypted with the entity key of the specific structured data entity; and
   the transmitting device of the processor configured to transmit the new first transport header and commit node pair of the specific structured data entity of the one or more structured data entities to one or more second computing devices in the network.
26. The system of any of the preceding items 16 to 25, comprising:
   the processor in the distributed network configured to:
      generate a new structured data entity data to be added to the database, the new structured data entity including data identifying an existing structured data entity of the one or more structured data entities to which the new structured data entity is to be associated, wherein the generating of the new structured data entity includes:
         generating, by the computing device in the network, a new first transport header node and new commit node pair, a new second transport header node and new encrypted relation node pair, and a new third transport header node and new encrypted data node pair;
         linking, by the computing device, the new structured data entity with the existing structured data entity; and
   the transmitting device of the processor configured to transmit the new structured data entity to one or more second computing devices in the network.
27. The system of any of the preceding items 16 to 26, wherein the new commit node includes the unique identifier of the existing structured data entity; and
   wherein the linking includes:
      inserting a hash of the encrypted relation node of the existing structured data entity in a blob reference of the new first transport header node.
28. The system of any of the preceding items 16 to 27, wherein each of the one or more structured data entities are associated with a medical patient.
29. The system of any of the preceding items 16 to 28, wherein the storage peer is associated with a dental office, and the producer peer is a computing device that generates dental data.
30. The system of any of the preceding items 16 to 29, wherein the encrypted data of the one or more structured data entities is stored encrypted in the database such that user of the database is permitted to only read the transport header nodes of the node pairs and the commit nodes without one or more decryption keys.
31. A method for distributed data storage and management of data, the method comprising:
   storing, by a computing device in the computing network, a data abstract interfaced with database, the data abstract storing one or more medical patient data files, each of the one or more medical patient data files including at least three entries:
      a first entry including a first section having a hash of a second section of the first entry and a hash of a third section of a second entry, and the second section of the first entry having at least a unique identifier, a digital signature, and a data key, the data key being a relation node key encrypted with an entity key specific to an associated medical patient file of the one or more medical patient data files;
      a second entry including a third section having a hash of an encrypted fourth section encrypted with the relation node key of the second entry and a hash of a fifth section of a third entry, and the fourth section of the second entry having a summary of data included in a third entry and a data decryption key for the third entry;
      a third entry including encrypted data related to the structured data entity encrypted using the data encryption key and the entity key specific to the associated medical patient data file;
   transmitting, by the computing device, a request for the first entry of a specific medical patient data file of the one or more medical patient data files; and
   receiving, by the computing device, the first entry of the specific medical patient data file
32. The method as in claim 31, comprising:
   storing, by the computing device, n first key, the first key granting access to one or more of a plurality of second keys, each of plurality of second keys associated with a specific medical patient data file of the one or more medical patient data files;
   identifying, by the computing device, a specific second key of the one or more second keys associated with the specific medial patient data file;
   decrypting, by the computing device, the data key using the specific second key;
   identifying, by the computing device, the second entry of the medical patient data file;
   decrypting, by the computing device, the fourth section of the second entry using the relation node key of the decrypted data key;
   identifying, by the computing device, the third entry of the medical patient data file; and
   decrypting, by the computing device, the encrypted data of the third entry of the medical patient data file using the data decryption key of the decrypted fourth section of the second entry.
33. A computer program product embodied in a non-transitory computer readable medium, the computer program product comprising computer readable program code being executable by a hardware data processor to cause the hardware data processor to perform the method of any of the claims 1 to 15.
34. A computer program product embodied in a non-transitory computer readable medium, the computer program product comprising computer readable program code being executable by a hardware data processor to cause the hardware data processor to perform the method of any of the claims 31 to 32.

1. A method for distributed storage and management of data, the method comprising:
receiving, by a computing device on a network, a set of dental data;
storing, by the computing device in the network, a data abstract interfaced with a database, the data abstract storing one or more structured data entities, each of the one or more structured data entities including at least three node pairs including:
a first transport header node paired with a commit node, the first transport header node including at least a blob reference to the commit node, one of the blob references of the commit node including a hash of the commit node and size of the commit node, and a second blob reference of the first transport header including a hash of a second transport header node and size of the second transport header, the commit node including a unique identifier, a digital signature, and a data key, the data key being a relation node key encrypted with an entity key specific to an associated structured data entity of the one or more structured data entities;
the second transport header node paired with an encrypted relation node encrypted with the relation node key, the second transport header including at least a blob reference to the encrypted relation node, one of the blob references of the encrypted relation node including a hash of a third transport header node and size of the third transport header, and a second blob reference of the second transport header including a hash of the encrypted relation node and size of the encrypted relation node, the encrypted relation node including a summary of data included in an encrypted data node and a data decryption key for the encrypted data node;
the third transport header node paired with the encrypted data node, the third transport header node including a blob reference of the data node, the blob reference of the data node including at least a size of the encrypted data node and a hash of the encrypted data node, wherein the encrypted data node includes encrypted data related to the associated structured data entity encrypted using the data encryption key;
transmitting, by the computing device, a request for the first transport header node and the paired commit node of a specific structured data entity of the one or more structured data entities; and
receiving, by the computing device, the first transport header node and the paired commit node of the specific structured data entity.

2. The method of claim 1, comprising:
storing, by the computing device, a user access key, the user access key granting permission to access one or more of a plurality of entity keys, each of the plurality of entity keys associated with a structured data entity of the one or more structured data entities;
identifying, by the computing device, a specific entity key of the one or more entity keys associated with the specific structured data entity;
decrypting, by the computing device, the data key using the specific entity key;
identifying, by the computing device, the second transport header node and the paired encrypted relation node based on the blob reference of the received commit node in the first transport header node;
decrypting, by the computing device, the encrypted relation node using the relation node key of the decrypted data key;
identifying, by the computing device, the third transport header node and the paired encrypted data node based on the encrypted relation node; and
decrypting, by the computing device, the encrypted data of the encrypted data node using the data decryption key of the decrypted relation node.

3. The method of claim 2, wherein the network includes one or more computing devices associated with one or more users, the one or more computing devices defined as at least one of: a storage peer, a producer peer, a proxy peer, or a backup peer; and
wherein the one or more users form one or more groups of users, each group of users associated with a user access key.

4. The method of claim 1, wherein the blob reference of the commit node in the first transport header node includes a hash of the commit node and the second blob reference in the first transport header node comprises at least a hash and size of a second transport header node of the associated structured data entity.

5. The method of claim 1, wherein the blob reference to the encrypted relation node in the second transport header node includes a hash of the encrypted relation node and the second blob reference in the second transport header node comprises at least one hash and size of at least one third transport header node of the associated structured data entity.

6. The method of claim 1, wherein the database is cloud storage.

7. The method of claim 1, wherein the network is a distributed network.

8. The method of claim 1, wherein the encrypted data is medical patient data.

9. The method of claim 1, wherein the node pairs of each of the one or more structured data entities form a directed acyclic graph.

10. The method of claim 1, comprising:
generating, by the computing device, a data update of encrypted data of a specific encrypted data node of a specific structured data entity of the one or more structured data entities;
generating, by the computing device, a new third transport header node paired with a new data node, the new third transport header node including at least a blob reference of the new data node comprising a hash and size of the new data node;
generating, by the computing device, a new data decryption key, the new data decryption key generated by hashing the new data node;
encrypting, by the computing device, the new data node with the new data encryption key;
generating, by the computing device, a new second transport header and a new relation node pair based on the new third transport header node and the new data node, wherein one or more blob references of the new second transport header for the new relation node includes at least a hash of the new third transport header node and a hash of the new relation node, the new relation node including a summary of the data update included in the encrypted new data node and the new data decryption key;
generating, by the computing device a new relation node key, the new relation node key generated by hashing the new relation node;
encrypting, by the computing device, the new relation node with the new relation node key;
generating, by the computing device, a new first transport header node and a new commit node pair based on the data update, wherein one or more blob references of the commit node in the new first transport header node includes at least a hash and size of at least one previously generated commit node of the associated structured data entity, and the new commit node includes a new data key, the new data key being the new relation node key encrypted with the entity key of the specific structured data entity; and
transmitting, by the computing device, the new first transport header and commit node pair of the specific structured data entity of the one or more structured data entities to one or more second computing devices in the network.

11. The method of claim 1, comprising:
generating, by the computing device, a new structured data entity data to be added to the database, the new structured data entity including data identifying an existing structured data entity of the one or more structured data entities to which the new structured data entity is to be associated, wherein the generating of the new structured data entity includes:
generating, by the computing device in the network, a new first transport header node and new commit node pair, a new second transport header node and new encrypted relation node pair, and a new third transport header node and new encrypted data node pair;
linking, by the computing device, the new structured data entity with the existing structured data entity; and
transmitting, by the computing device, the new structured data entity to one or more second computing devices in the network.

12. The method of claim 11, wherein the new relation node and its transport header include the unique identifier of the existing structured data entity; and
wherein the linking includes:
inserting a hash and size of the transport header of the commit node of the existing structured data entity into the transport header of the new relation node.

13. The method of claim 1, wherein each of the one or more structured data entities are associated with a dental patient.

14. The method of claim 3, wherein the storage peer is associated with a dental office, and the producer peer is a computing device that generates dental data.

15. The method of claim 1, wherein the encrypted data of the one or more structured data entities is stored in the database such that the user of the database is able to only read the transport header nodes of the node pairs and the commit nodes without one or more decryption keys.
